(19) United States
(12) Patent Application Publication
(10) Pub. No.: US 2015/0080786 A1
(43) Pub. Date: Mar. 19, 2015

(54) Compact Consciousness Arousing Device

(71) Applicants: Robert E. King, Hawthorne, NV (US); Tina A. Manzini, Hawthorne, NV (US)

(72) Inventors: Robert E. King, Hawthorne, NV (US); Tina A. Manzini, Hawthorne, NV (US)

(21) Appl. No.: 14/468,870

(22) Filed: Aug. 26, 2014

Related U.S. Application Data
(60) Provisional application No. 61/878,339, filed on Sep. 16, 2013.

(51) Int. Cl. A61M 21/00; A61M 31/00

(52) U.S. Cl. CPC A61M 21/00; A61M 31/00; A61M 2021/0016; A61M 2021/0077; A61M 2202/02
USPC 604/26

(57) ABSTRACT

A pen-shaped device has a trigger mechanism placed within a trigger housing, the trigger mechanism being used to puncture an adjacent gas containing ampoule housed in an ampoule receptacle. The trigger mechanism is actuated by a cap, which is connected to a stop (the latter connected to a plunger) of the trigger mechanism. A prong is provided at the bottom of the plunger, enabling the trigger mechanism to puncture the gas containing ampoule. A spring is connected to the trigger housing and provides resistance against actuation, ensuring the gas containing ampoule is not accidently punctured. In order replace expended gas containing ampoule, the ampoule receptacle can be unscrewed from the trigger housing. The expended ampoule can be removed and a new ampoule installed. A clip is connected to the cap, allowing the present invention to be securely carried, primarily by being attached to a pocket.

The current application claims a priority to the U.S. Provisional Patent application Ser. No. 61/878,339 filed on Sep. 16, 2013.

### FIELD OF THE INVENTION

The present invention relates generally to medical smelling salts ampoules. More specifically, the present invention is a compact consciousness arousing device which is designed to make smelling salts ampoules more handy to medical practitioners and patients alike. The present invention is a pen shaped instrument which accepts a single smelling sat ampoule within its body. The ampoule can be broken, releasing its active ingredients, by simply pushing down on the top of the pen structure. The active ingredients of the ampoule are allowed to escape the pen structure by way of a plurality of ventilation holes near its tip.

### BACKGROUND OF THE INVENTION

Humankind has always suffered from a myriad of different medical problems ranging from physical injuries to diseases caused by pathogens and genetic defects. Many physical injuries and diseases can be life threatening, and can cause great pain and suffering in those people who are afflicted by them. Compassionate people have toiled for centuries in an effort to cure various disease and develop more efficient ways of mending a physically damaged human body. As a result of this ongoing effort, humanity has accomplished the development and implementation of new medical methods and technology. Although there is much that we still do not understand, or have the capability to cure or heal, the field of medicine has certainly advanced substantially as it has passed down through the centuries becoming what we know and accept as the modern field of medicine.

There are a great many methods and technologies which are associated with the field of medicine, and all of them have some specific purpose. One such technology is known as the ampoule. Ampoules are essentially small glass containers which are hermetically sealed off from the external environment. The ampoule can be opened by snapping off the very top of the device; this usually produces a clean break with no glass shards being dispersed into the liquid contained within the ampoule. The primary purpose of an ampoule is the containment of substances which may be compromised by contact with the external air or any other impurities. The medical field makes extensive use of fluid pharmaceuticals which are often injected directly into the bloodstream of a patient. It is imperative that these pharmaceuticals are protected from any external contamination in order to maintain their effectiveness and ensure that no contaminants are introduced into the bloodstream of the patient when the pharmaceutical is injected. Ampoules are also extremely useful for the containment of chemicals and chemical compounds which may react with the ambient atmosphere. In such cases the chemicals or chemical compounds must be kept in a container which is hermetically sealed, and therefore impermeable to gas or fluid transfer across its walls. Ampoules are ideal for this particular use and, as a result, still see widespread use in the modern world, particularly in the fields of medicine and chemistry.

Historically, ampoules were used in religious purposes; a small amount of a person's blood was taken after their death and stored within a glass ampoule. This glass ampoule was then buried with the deceased individual. This tradition was commonly practiced for those who were entombed within Christian catacombs. Of course in the modern world, ampoules typically serve containment purposes in the medical and pharmaceutical fields as mentioned above. One specific modern day use of ampoules is for the containment of smelling salts. Smelling salts are a particular chemical compound which is useful in arousing consciousness in humans through stimulation of the sympathetic nervous system. This stimulation is caused by a small amount of ammonia gas which is released by the smelling salts and inhaled by the patient being treated with the smelling salts. In the past, smelling salts saw widespread use in order to help treat fainting, and were often carried by constabulary forces and found in first aid kits in workplaces. In the modern world, the usage of smelling salts has decreased significantly, and is now typically only used in hospitals, by professional athletes, and by patients who would suffer serious ill effects by suddenly losing consciousness.

Although the widespread use of smelling salts has decreased significantly, they are still used in hospital settings to arouse consciousness in patients, and are sometimes used by those who are prone to fainting. The decreased use of smelling salts ampoules has led to them being less and less often kept on hand by medical practitioners. As a result, the old adage of when something is need it is not present applies. Often when a smelling salts ampoule is needed by a medical practitioner it is not available on hand. In such situations the smelling salts ampoules must be retrieved from a storage closet, causing significant delay in the time between the medical practitioner determining the need for a smelling salts ampoule, and the actual administration of the smelling salts ampoule. At best this delay is merely an annoyance and at worse the delay could have serious repercussions for the patient. It is clear that there is a potential for a device which enables smelling salts ampoules to be kept handy by a medical practitioner, or by a patient who is recommended to keep them on hand.

It is an object of the present invention to introduce an apparatus which greatly increases the portability, handiness, and usability of smelling salts ampoules. It is an object of the present invention to utilize a structure similar to a pen which accepts any standard smelling salts ampoule into its interior. It is a further object of the present invention to provide a mechanism by which the ampoule may be broken open, thereby releasing its active ingredients to arouse consciousness in a patient; the active ingredients are allowed to escape via a plurality of ventilation holes near the tip of the apparatus. It is an additional object of the present invention to be refillable such that it may be reused; only the ampoule itself must be replaced after use, and this is enabled by allowing the present invention to be opened to insert a fresh ampoule when need be.

### BRIEF DESCRIPTION OF THE DRAWINGS

FIG. 1 is an exploded view of the present invention.

FIG. 2 is a perspective view of the present invention.

FIG. 3 is a front view thereof, showing the plane upon which a cross-sectional view is taken and shown in FIG. 4.

FIG. 4 is a perspective cross-sectional view thereof, taken along line A-A of FIG. 3.

FIG. 5 is a front view of a trigger housing of the present invention, showing the plane upon which a cross-sectional view is taken and shown in FIG. 6.

FIG. 6 is a perspective cross-sectional view thereof, taken along line A-A of FIG. 5.

FIG. 7 is a front view of a cap and the trigger mechanism of the present invention, showing the plane upon which a cross-sectional view is taken and shown in FIG. 8.

FIG. 8 is a perspective cross-sectional view thereof, taken along line A-A of FIG. 7.

FIG. 9 is a front view of an ampoule receptacle of the present invention, showing the plane upon which a cross-sectional view is taken and shown in FIG. 10.

FIG. 10 is a perspective cross-sectional view thereof, taken along line A-A of FIG. 9.

### DETAIL DESCRIPTIONS OF THE INVENTION

All illustrations of the drawings are for the purpose of describing selected versions of the present invention and are not intended to limit the scope of the present invention.

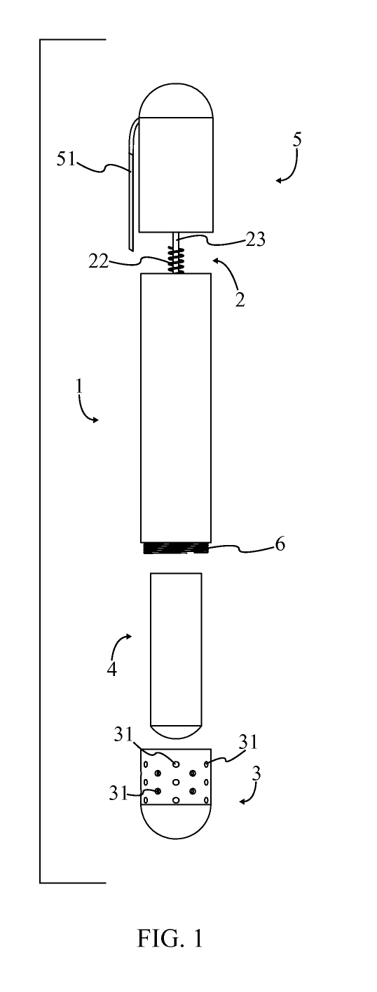

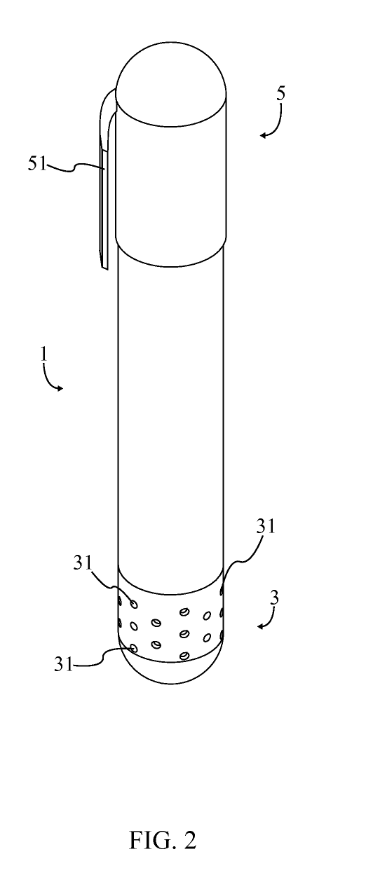

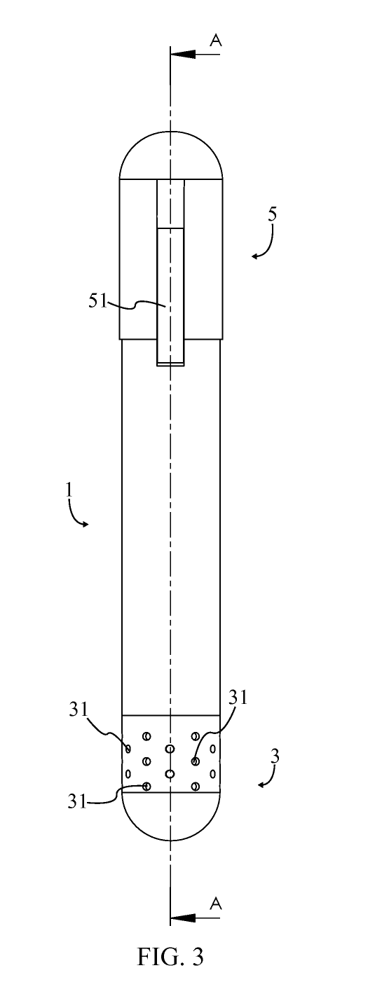

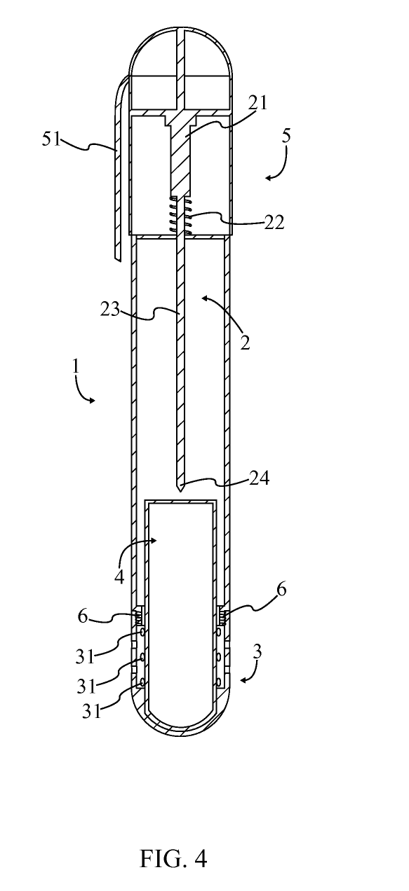

The present invention is a compact consciousness arousing device, allowing it to easily be carried on one's person and quickly accessible if needed to restore consciousness to an unconscious individual. Especially beneficial in hospital settings, the present invention is easily and quickly utilized, in part due to its compact nature. The present invention comprises a trigger housing 1, a trigger mechanism 2, an ampoule receptacle 3, a gas containing ampoule 4, and a cap 5. The trigger housing 1 forms the main body of the present invention, with the ampoule receptacle 3 being positioned at one end of the trigger housing 1 and the cap 5 being positioned at the other end of the trigger housing 1. The gas containing ampoule 4 is housed in the ampoule receptacle 3, where it can easily be punctured by the trigger mechanism 2. The cap 5 is connected to the trigger mechanism 2, allowing a user to engage the trigger mechanism 2 by simply applying pressure to the cap 5. In the preferred embodiment the present invention resembles a pen, with the trigger housing 1 having a cylindrical shape and the ampoule receptacle 3 tapering away from the trigger housing 1. A quantity of smelling salts 41 is stored in the gas containing ampoule 4. When the gas containing ampoule 4 is punctured, a plurality of ventilation holes 31 on the ampoule receptacle 3 allow the smelling salts 41 to be dispersed such that an unconscious individual can be awakened. The positioning and relations of these components is shown through FIG. 1, FIG. 2, FIG. 3, and FIG. 4.

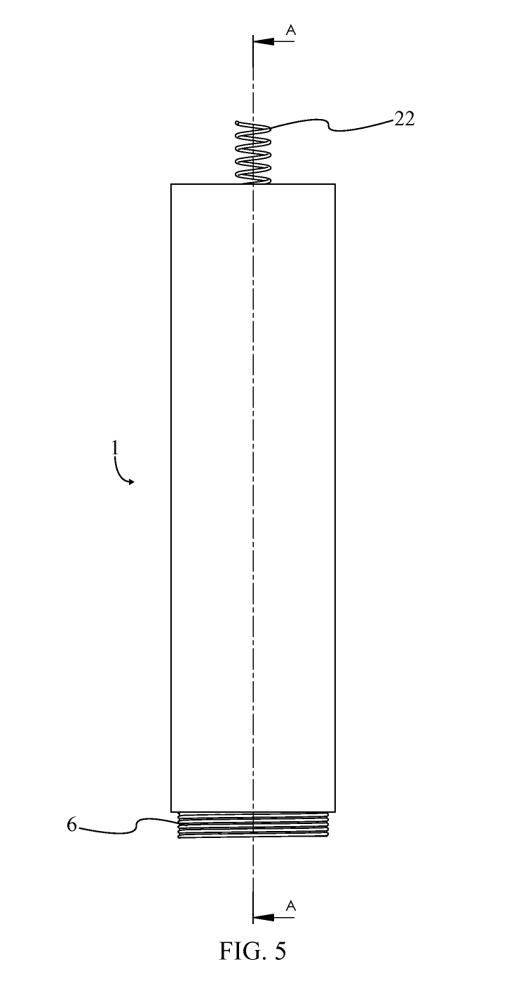

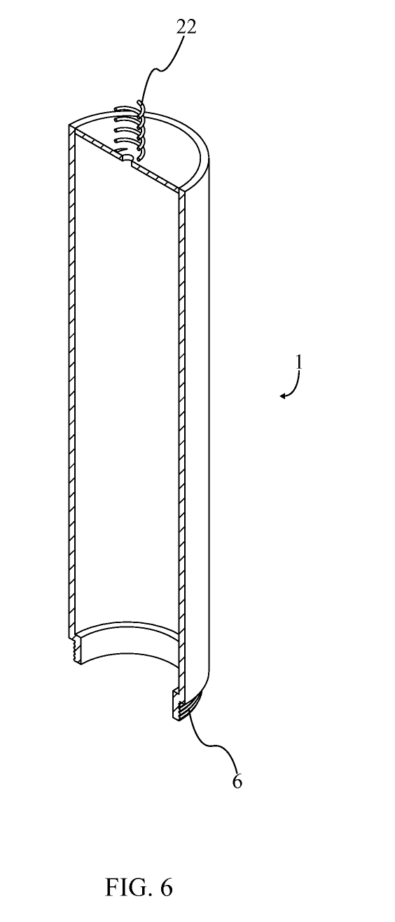

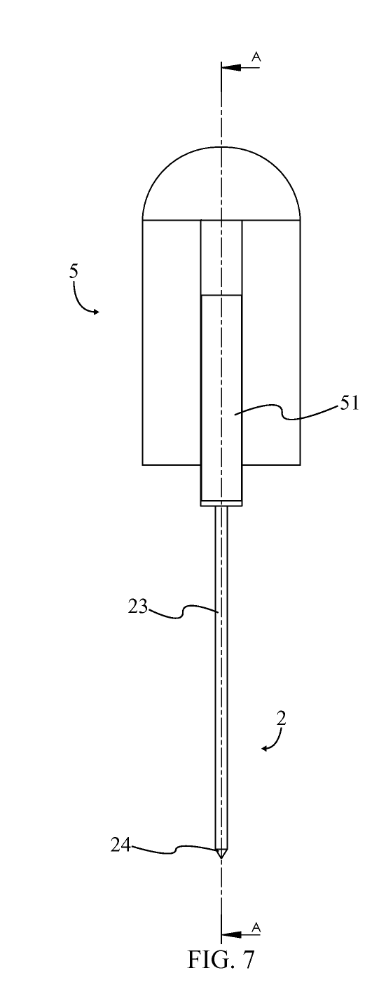

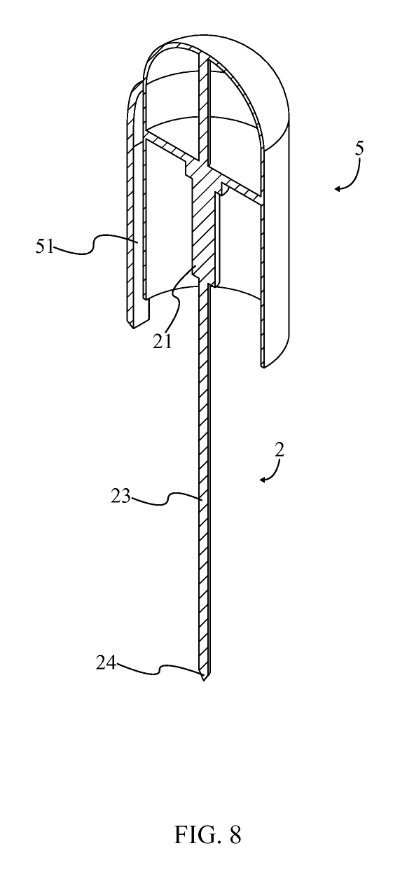

The trigger mechanism 2 comprises a stop 21, a spring 22, a plunger 23, and a prong 24. The stop 21 is connected to both the cap 5 and the plunger 23, and is positioned adjacent to the spring 22. The stop 21 serves to restrict linear motion of the cap 5 (in combination with the spring 22), and thus the connected plunger 23. This prevents the trigger mechanism 2 from traversing too far into the gas containing ampoule 4 when engaged. The stop 21 and spring 22 also provide resistance when the cap 5 is pressed; this helps ensure that the cap 5 is not accidently pressed, resulting in unintentional puncturing of the gas containing ampoule 4. By raising the minimum force (i.e. via the stop 21 and spring 22) required to engage the trigger mechanism 2 the present invention protects against accidental discharge. The spring 22 is connected to the trigger housing 1, such that it can resist movement of the stop 21 and thus cap 5. The prong 24 is connected to the plunger 23 opposite the stop 21. The prong 24 is thus positioned adjacent to the ampoule, such that it can easily puncture the ampoule when the trigger mechanism 2 is engaged. Parts of the trigger mechanism 2 can be seen in FIG. 1, FIG. 4, FIG. 5, FIG. 6, FIG. 7, and FIG. 8.

The prong 24 is preferably a sharpened point that can easily puncture the gas containing ampoule 4. To increase puncture capability, a plurality of prongs 24 may instead be utilized, providing more contact points which can break the surface of the gas containing ampoule 4.

The cap 5, which actuates the trigger mechanism 2, is sleeved over the trigger housing 1. To allow the movement of the cap 5, the cap 5 is slidably engaged with the trigger housing 1. A user can push the cap 5 along the trigger housing 1, towards the ampoule receptacle 3. As the cap 5 is pressed, it causes corresponding movement of the connected stop 21, plunger 23, and prong 24. This movement brings the prong 24 into contact with the gas containing ampoule 4, allowing the prong 24 to puncture the gas containing ampoule 4. The cap 5 can be seen in FIG. 1, FIG. 2, FIG. 3, FIG. 4, FIG. 7, and FIG. 8.

As a user presses against the cap 5 and causes movement of the trigger mechanism 2, the stop 21 comes into contact with the spring 22. As the stop 21 presses against the spring 22, the spring 22 is compressed and creates an opposing force that impedes the stop 21 and which ultimately prevents the cap 5 from moving past a terminal point. The spring 22 also returns the stop 21 and cap 5 to an equilibrium position when a user ceases to press on the cap 5; in effect, thus a user does not have to manually reset the trigger mechanism 2 after use.

To help secure the present invention to various articles (e.g. a shirt pocket), a clip 51 is externally connected to the cap 5. The clip 51 enables the present invention to be securely carried (e.g. there is a minimal risk of falling out of the shirt pocket) while still being in an easily accessed location. Ease of access is important as it minimizes reaction time when attempting to treat an unconscious person.

The gas containing ampoule 4 can potentially be attached to the ampoule receptacle 3 for a more secure configuration. While the gas containing ampoule 4 can normally be held in place by means of an interference fit, or prevented from moving by the adjacent trigger mechanism 2, it may be desirable to physically secure the gas containing ampoule 4 to the ampoule receptacle 3. One way this can be accomplished is by providing matching threading within the ampoule receptacle 3 and on the gas containing ampoule 4, allowing the gas containing ampoule 4 to be screwed into the ampoule receptacle 3. Other attachment methods are also possible, some additional non-limiting examples include a latch mechanism and a clamp. The gas containing ampoule 4 is shown in FIG. 1 and FIG. 4.

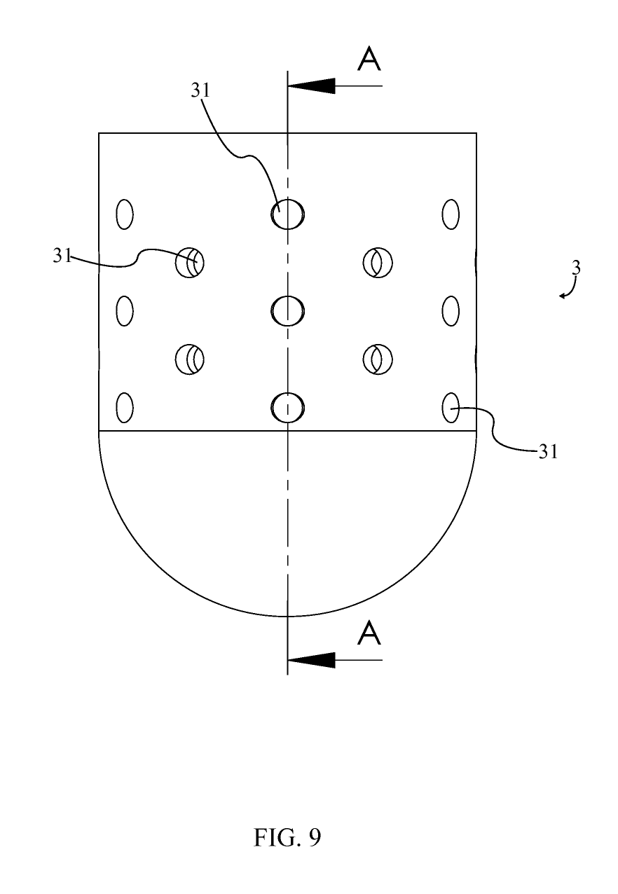

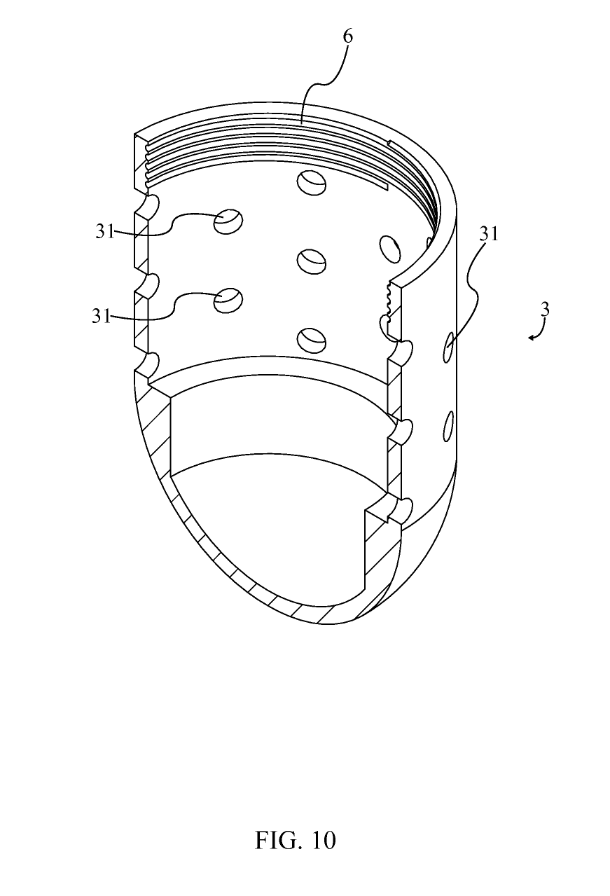

To allow the contents of the gas containing ampoule 4 to be dispersed, the ampoule receptacle 3 comprises a plurality of ventilation holes 31. These ventilation holes 31 traverse through ampoule receptacle 3, distributed around the ampoule receptacle 3 to allow content dispersion in all directions. The ventilation holes 31 provide a direct path between the interior of the present invention and an exterior area; this is necessary to allow the ampoule contents to easily be dispensed to an individual in need. The ampoule receptacle 3 is shown in FIG. 1, FIG. 2, FIG. 3, FIG. 4, FIG. 9, and FIG. 10.

The quantity of smelling salts 41, while an integral part of the present invention, is not limited to a specific formulation. Any formulation which induces consciousness in an individual can be used for the quantity of smelling salts 41. In the preferred embodiment, the quantity of smelling salts 41 is an ammonia based compound. As ammonia based compounds are traditionally used for smelling salts 41, they are readily available and inexpensive compared to potential rarer and less utilized formulations. The preferred choice of an ammonia based compound does not preclude the use of alternative formulations in different embodiments of the present invention. If an alternative formulation is utilized, it should be one that is safely stored within an ampoule and which can quickly be dispersed through the plurality of ventilation holes 31 in order to arouse consciousness.

While the gas containing ampoule 4 is generally not reusable, the present invention is designed to accommodate replacement gas containing ampoules 4. This lowers cost and waste as a user can simply replace the gas containing ampoule 4 after each use, as opposed to having to replace the entire present invention. Supporting the provision of replacement ampoules, the ampoule receptacle 3 can be detached from the trigger housing 1, allowing the gas containing ampoule 4 to be accessed and replaced. While a number of attachment methods are possible, the trigger housing 1 screws into the ampoule receptacle 3 in the preferred embodiment. Resultantly, a threaded portion 6 is provided on both the trigger housing 1 and the ampoule receptacle 3, with these threaded portions 6 being engaged with each other in order to join the trigger housing 1 and ampoule receptacle 3.

Though the preferred embodiment uses a threading-based attachment, other embodiments can use different mechanisms to attach the trigger housing 1 and the ampoule receptacle 3 to each other. For example, a snap fit, press fit, or even paired magnets could be used in place of the preferred embodiment's threading.

As an accessory, the present invention can be distributed in a package that includes a plurality of replacement gas containing ampoules 4. The packaging may be used to store the present invention prior to use, whether for retail display or general inventory management. Providing a retail package that has both the present invention and replacement ampoules allows potential customers to procure and use the present invention without having to immediately account for replacements. Replacement ampoules can also be provided separately, such that they can be purchased independently by those who already own the present invention.

As earlier referenced, the present invention is preferably configured to resemble a pen. This is because pens, especially in hospital settings, are commonly carried by medical professionals and having an extra pen is unlikely to be burdensome. Pens are also compact and easily carried, increasing user-friendliness. These facets of the present invention, the compact size and ease of use, are important as they help increase adoption by medical professionals; if a tool is so cumbersome or difficult to use that a professional does not carry the tool, the tool loses its value. That being said, the present invention is primarily provided for the housing and breaking of a gas containing ampoule 4. Thus, in other embodiments, the present invention may be configured in a way that does not resemble a pen. Though the appearance of such alternative embodiments may vary, the components and their interactions remain similar to those of the preferred embodiment. Similarly, dimensions of the present invention may be varied without negatively impacting the intended functions.

Although the invention has been explained in relation to its preferred embodiment, it is to be understood that many other possible modifications and variations can be made without departing from the spirit and scope of the invention as hereinafter claimed.

What is claimed is:

1. The Compact Consciousness Arousing Device comprises:
a trigger housing;
a trigger mechanism;
the trigger mechanism comprises a stop, a spring, a plunger, and a prong;
an ampoule receptacle;
the ampoule receptacle comprises a plurality of ventilation holes;
a gas containing ampoule;
a cap;
the trigger mechanism being positioned into the trigger housing;
the trigger mechanism being positioned between the cap and the gas containing ampoule;
the stop being adjacently connected between the cap and the plunger;
the stop being positioned adjacent to the spring;
the prong being connected to the plunger opposite the stop;
the ampoule receptacle being adjacently attached to the trigger housing;
the gas containing ampoule being housed in the ampoule receptacle; and
the plurality of ventilation holes traversing through the ampoule receptacle.

2. The Compact Consciousness Arousing Device as claimed in claim 1 comprises:
the spring being mounted to the trigger housing;
the spring being adjacently positioned to the stop opposite the cap; and
the prong being positioned adjacent to the gas containing ampoule.

3. The Compact Consciousness Arousing Device as claimed in claim 2 comprises:
the trigger housing being sleeved into the cap; and
the cap being slidably engaged with the trigger housing.

4. The Compact Consciousness Arousing Device as claimed in claim 1 comprises:
the plurality of ventilation holes being distributed around the ampoule receptacle.

5. The Compact Consciousness Arousing Device as claimed in claim 1 comprises:
the trigger housing and the ampoule receptacle each comprise a threaded portion; and
the threaded portion of the trigger housing being helically engaged with the threaded portion of the ampoule receptacle.

6. The Compact Consciousness Arousing Device as claimed in claim 1 comprises:
a clip; and
the clip being externally connected to the cap.

7. The Compact Consciousness Arousing Device as claimed in claim 1 comprises:
a quantity of smelling salt; and
the smelling salt being located within the gas containing ampoule.

8. The Compact Consciousness Arousing Device as claimed in claim 7 comprises:
the smelling salt being an ammonia-based compound.

9. The Compact Consciousness Arousing Device comprises:
a trigger housing;
a trigger mechanism;
the trigger mechanism comprises a stop, a spring, a plunger, and a prong;
an ampoule receptacle;
the ampoule receptacle comprises a plurality of ventilation holes;
a gas containing ampoule;
a cap;
a clip;
a quantity of smelling salt;
the trigger mechanism being positioned into the trigger housing;
the trigger mechanism being positioned between the cap and the gas containing ampoule;
the stop being adjacently connected between the cap and the plunger;
the stop being positioned adjacent to the spring;
the prong being connected to the plunger opposite the stop;
the ampoule receptacle being adjacently attached to the trigger housing;
the gas containing ampoule being housed in the ampoule receptacle; and
the plurality of ventilation holes traversing through the ampoule receptacle.

10. The Compact Consciousness Arousing Device as claimed in claim 9 comprises:
the spring being mounted to the trigger housing;
the spring being adjacently positioned to the stop opposite the cap;
the prong being positioned adjacent to the gas containing ampoule;
the trigger housing being sleeved into the cap; and
the cap being slidably engaged with the trigger housing.

11. The Compact Consciousness Arousing Device as claimed in claim 9 comprises:
the plurality of ventilation holes being distributed around the ampoule receptacle.

12. The Compact Consciousness Arousing Device as claimed in claim 9 comprises:
the trigger housing and the ampoule receptacle each comprise a threaded portion; and
the threaded portion of the trigger housing being helically engaged with the threaded portion of the ampoule receptacle.

13. The Compact Consciousness Arousing Device as claimed in claim 9 comprises:
the clip being externally connected to the cap.

14. The Compact Consciousness Arousing Device as claimed in claim 9 comprises:
the smelling salt being located within the gas containing ampoule; and
the smelling salt being an ammonia-based compound.

15. The Compact Consciousness Arousing Device comprises:
a trigger housing;
a trigger mechanism;
the trigger mechanism comprises a stop, a spring, a plunger, and a prong;
an ampoule receptacle;
the ampoule receptacle comprises a plurality of ventilation holes;
a gas containing ampoule;
a cap;
a clip;
a quantity of smelling salt;
the trigger mechanism being positioned into the trigger housing;
the trigger mechanism being positioned between the cap and the gas containing ampoule;
the stop being adjacently connected between the cap and the plunger;
the stop being positioned adjacent to the spring;
the prong being connected to the plunger opposite the stop;
the ampoule receptacle being adjacently attached to the trigger housing;
the trigger housing and the ampoule receptacle each comprise a threaded portion;
the threaded portion of the trigger housing being helically engaged with the threaded portion of the ampoule receptacle;
the gas containing ampoule being housed in the ampoule receptacle;
the plurality of ventilation holes traversing through the ampoule receptacle; and
the smelling salt being located within the gas containing ampoule.

16. The Compact Consciousness Arousing Device as claimed in claim 15 comprises:
the spring being mounted to the trigger housing;
the spring being adjacently positioned to the stop opposite the cap;
the prong being positioned adjacent to the gas containing ampoule;
the trigger housing being sleeved into the cap; and
the cap being slidably engaged with the trigger housing.

17. The Compact Consciousness Arousing Device as claimed in claim 15 comprises:
the plurality of ventilation holes being distributed around the ampoule receptacle.

18. The Compact Consciousness Arousing Device as claimed in claim 15 comprises:
the clip being externally connected to the cap.

19. The Compact Consciousness Arousing Device as claimed in claim 15 comprises:
the smelling salt being an ammonia-based compound.
